(19) United States
(12) Patent Application Publication
(10) Pub. No.: US 2025/0345494 A1
(43) Pub. Date: Nov. 13, 2025

(54) APHERESIS REGISTRATION SYSTEM

(71) Applicant: Haemonetics Corporation, Boston, MA (US)

(72) Inventor: Melvin Tan, Boston, MA (US)

(21) Appl. No.: 19/202,521

(22) Filed: May 8, 2025

Related U.S. Application Data
(60) Provisional application No. 63/644,205, filed on May 8, 2024.

(51) Int. Cl. A61M 1/02; G16H 40/40

(52) U.S. Cl. CPC A61M 1/0259; G16H 40/40; A61M 2205/3553; A61M 2205/3592; A61M 2209/08

(57) ABSTRACT

A portable apheresis system for separating blood into blood components includes a cellular modem configured to transmit and receive information to and from the apheresis device via a cellular connection, and a connector operatively coupled with the cellular modem. The connector is configured to control the cellular modem to transmit and receive apheresis device registration information, establish, using the cellular modem, a cellular communication connection between an apheresis device and a cellular network after detecting the cellular network, transmit registration information relating to the apheresis device to a registration and connectivity computer software server via the wireless communication connection, receive an apheresis registration response from the registration and connectivity computer software server, the apheresis registration response including information relating to the apheresis device registration process, the registration process causing direct or indirect apheresis device communication with a blood establishment computer system remotely positioned relative to the apheresis device.

### CROSS REFERENCE TO RELATED APPLICATIONS

This application claims the benefit of U.S. Provisional Patent Application No. 63/644,205, filed May 8, 2024, the contents of which are incorporated by reference herein in its entirety as if fully set forth.

### FIELD

Illustrative embodiments of the invention generally relate to blood processing and, more particularly, various embodiments of the invention relate to electronically registering and communicating apheresis systems.

### BACKGROUND

A pheresis devices are widely used to separate whole blood into individual components. Typically, these devices are networked with remote devices across a wide network, such as the Internet. To make that connection, a technician configures and/or programs the apheresis device to communicate with a remote device, such as remote server running device registration and connectivity computer software. In the case of plasma centers/networks, the number of apheresis devices can reach into the hundreds or even thousands, resulting in a considerable burden of configuration processes. This proliferation inevitably imposes a significant strain on available resources. For example, among other things, this proliferation expands time-consuming efforts of device registration.

### SUMMARY OF VARIOUS EMBODIMENTS

In accordance with one embodiment, a portable apheresis system for separating blood into blood components includes a cellular modem configured to transmit and receive information to and from the apheresis device via a cellular connection, and a connector operatively coupled with the cellular modem. The connector is configured to control the cellular modem to transmit and receive apheresis device registration information, establish, using the cellular modem, a cellular communication connection between an apheresis device and a cellular network after detecting the cellular network, transmit registration information relating to the apheresis device to a registration and connectivity computer software server via the wireless communication connection, receive an apheresis registration response from the registration and connectivity computer software server, the apheresis registration response including information relating to the apheresis device registration process, the registration process causing direct or indirect apheresis device communication with a blood establishment computer system remotely positioned relative to the apheresis device.

In some embodiments, the registration information comprises one or more of a serial identifier, location information, timestamps, component versions, procedure data, user information, a device activation code, licensing, authorized use information, or location specific information.

In some embodiments, the apheresis device is validated based upon one or more of the following: the apheresis device is valid, licensed, and intended for use to whom it was sold or placed, or determining that a geographic area of use is valid for the apheresis device.

In some embodiments, the connector is configured to receive registration confirmation information from the registration and connectivity computer software server via the cellular connection, the apheresis device being prevented from operating when validation fails or when the apheresis device is not registered.

In some embodiments, the registration information includes one or more of the following: information about the apheresis device, identification data or device data.

In some embodiments, the identification data includes one or more of the following: a serial identifier, location, timestamps, or component versions.

In some embodiments, the device data includes one or more of the following: procedure data, user data or location specific information.

In some embodiments, the registration process is plug-and-play.

In some embodiments, the system further includes a mobile platform containing the apheresis device, the mobile platform being towable or self-propelled.

In accordance with one embodiment, a method for separating blood into blood components includes transmitting and receiving, via a cellular modem, information to and from the apheresis device via a cellular connection; controlling the cellular modem to transmit and receive apheresis device registration information; establishing, by the connector, using the cellular modem, a cellular communication connection between the apheresis device and a cellular network after detecting the cellular network; transmitting registration information relating to the apheresis device to a registration and connectivity computer software server via the wireless communication connection; and receiving an apheresis registration response from the registration and connectivity computer software server, the apheresis registration response including information relating to the apheresis device registration process, the registration process causing direct or indirect apheresis device communication with a blood establishment computer system (BECS) remotely positioned relative to the apheresis device.

In some embodiments, the registration information comprises one or more of a serial identifier, location information, timestamps, component versions, procedure data, user information, a device activation code, licensing, authorized use information, or location specific information.

In some embodiments, the method further includes validating, by the registration and connectivity computer software server based upon one or more of the following: the apheresis device is valid, licensed, and intended for use to whom it was sold or placed, or determining that a geographic area of use is valid for the apheresis device.

In some embodiments, the registration response is received from the registration and connectivity computer software server via the cellular connection, the apheresis device being prevented from operating when validation fails or when the apheresis device is not registered.

In some embodiments, the registration information includes one or more of the following: information about the apheresis device, identification data or device data.

In some embodiments, the identification data includes one or more of the following: a serial identifier, location, timestamps, or component versions.

In some embodiments, the device data includes one or more of the following: procedure data, user data or location specific information.

Illustrative embodiments are implemented as a computer program product having a computer usable medium with computer readable program code thereon. The computer readable code may be read and utilized by a computer system in accordance with conventional processes.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 schematically shows a network communicating a plurality of apheresis devices with a registration server and BECS in accordance with illustrative embodiments.

FIG. 2 schematically shows a perspective view of a blood processing system in accordance with illustrative embodiments.

FIG. 3 schematically shows a plan view of the blood processing system of FIG. 2.

FIG. 4 schematically shows an example of a disposable set installed within the blood processing system of FIG. 2 in accordance with illustrative embodiments.

FIG. 5 schematically shows selected parts of an apheresis device configured in accordance with illustrative embodiments.

FIG. 6 schematically shows selected parts of a registration server configured in accordance with illustrative embodiments.

FIG. 7 shows a process of registering an apheresis device in accordance with illustrative embodiments.

FIG. 8 is a diagram of an example embodiment of signals and operations among an apheresis system, a registration and connectivity computer software server and a BECS in accordance with illustrative embodiments.

FIG. 9 is an example block diagram of a device in accordance with illustrative embodiments.

### DESCRIPTION OF ILLUSTRATIVE EMBODIMENTS

In illustrative embodiments, an apheresis device is configured to be effectively used in an environment that does not have extensive communication infrastructure, such as a mobile environment or a temporary building. Such embodiments may more easily register the apheresis device with its underlying blood management network (e.g., a plasma or apheresis network). To those ends, the apheresis device may include wireless (e.g., cellular, 802.xx, etc.) communication connectivity to enable use in the mobile environment, as well as “plug-and-play” functionality to automatically register in its network with minimal or no manual interaction. Details of illustrative embodiments are discussed below.

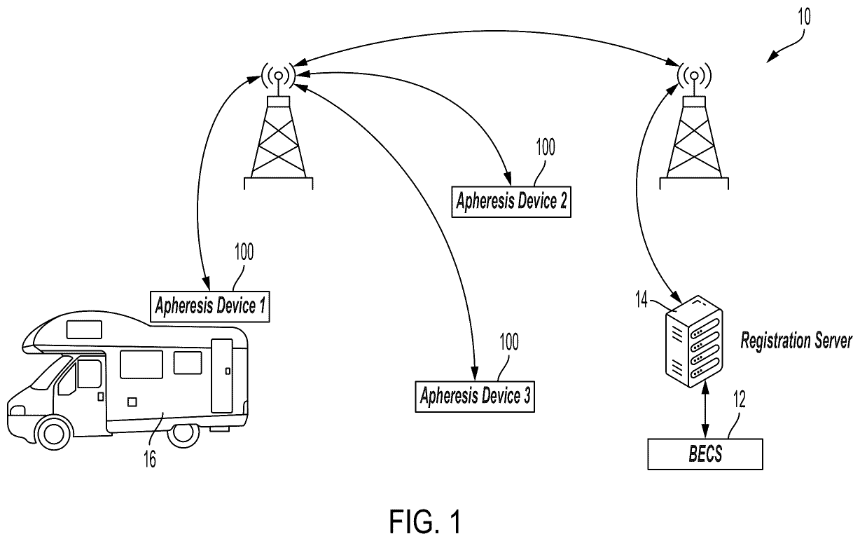

FIG. 1 schematically shows a blood management network (“network 10”) communicating a plurality of apheresis devices 100 with a registration server 14 in accordance with illustrative embodiments. In this example, three apheresis devices 100 operate within the same functional or business unit and are controlled and coordinated by a set of one or more remote device registration servers 14. Among other things, the functional/business entity operating the apheresis devices 100 may be a single plasma center. As known by those in the art and discussed in greater detail below, the registration server 14 manages data flow and registration with the apheresis devices 100 and communication with other electronic management systems, such as a remotely located blood establishment computer system (“BECS 12”).

Traditionally, apheresis devices have been located in “brick-and-mortar” locations, such as stationary buildings. These locations (or multiple co-owned or co-managed locations) often have hundreds or even thousands of apheresis devices 100. Accordingly, remote apheresis devices 100 may be adaptable to provide efficient networked use in the field.

The inventors discovered this problem. Accordingly, in illustrative embodiments, some or all of the apheresis devices 100 of various embodiments are configured to operate on a mobile platform 16, such as a self-propelled vehicle (e.g., a truck, van, etc.) or a trailer. As such, these apheresis devices 100 are specially configured to communicate with remote network devices using wireless communication technologies that do not require a static facility. For example, FIG. 1 shows apheresis devices 100 (e.g., designated apheresis device 1, apheresis device 2, and apheresis device 3) communicating with a serving radio access network (RAN) using 3rd Generation Partnership Project (3GPP) cellular communication technology, such as LTE or 5G (or future cellular technology, such as the potential 5G successor technology known as “6G”). Other embodiments, however, may communicate with other similarly untethered wireless network communication technologies, such as satellite communication networks, Institute of Electrical and Electronics Engineers (IEEE) 802.xx networks, WiMAX networks, mesh networks, point-to-point or point-to-multipoint wireless networks, fixed wireless access, and/or microwave links.

As shown in FIG. 1, the registration server 14 is also in communication with a different RAN than the apheresis devices 100. However, those skilled in the art may appreciate that the registration server 14 and apheresis devices 100 may be served by a same RAN or base station (e.g., gNodeB, etc.).

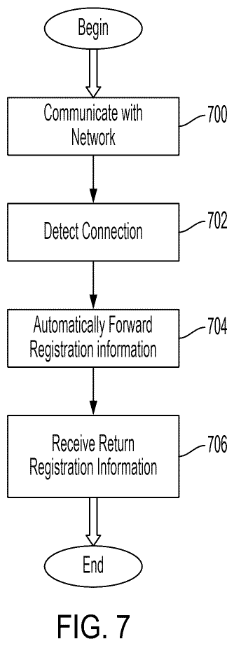

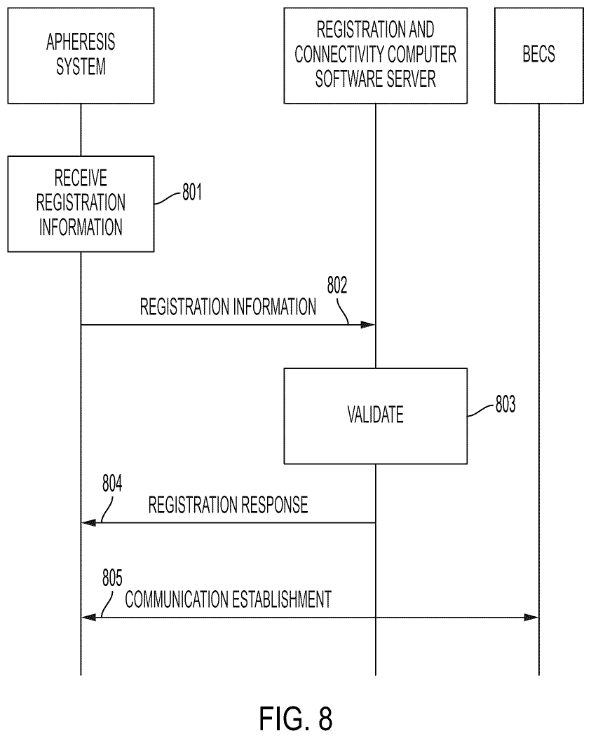

Additional problems arise when updating, replacing, changing over, or adding apheresis devices 100 to the business or entity managing the devices (e.g., a plasma center). Specifically, before the apheresis device 100 can connect to the system, a technician or other system user manually registers the device 100, which typically requires significant time and consequential cost. In addition to the time required to register, this often requires travel expenses for a highly trained, highly paid technician to travel to the center with the apheresis device(s) 100. This manual process can be especially costly, for example, when changing over or replacing an entire plasma center with thousands of apheresis devices 100. To obviate this problem, as discussed below in greater detail with regard to FIGS. 7 and 8, the apheresis devices 100 and registration servers 14 are configured to automatically register a new or updated apheresis device 100 with the network 10 upon connection to the network 10.

Some embodiments, however, may require minimal interaction, such as approving the registration by accepting certain safety messages (e.g., a button displaying “click ok to register”) after connection. In either case, in some embodiments, even these minimal requirements may be considered part of the registration process. Some embodiments, however, do not have these minimal requirements and registration is initiated and completed without any user interaction.

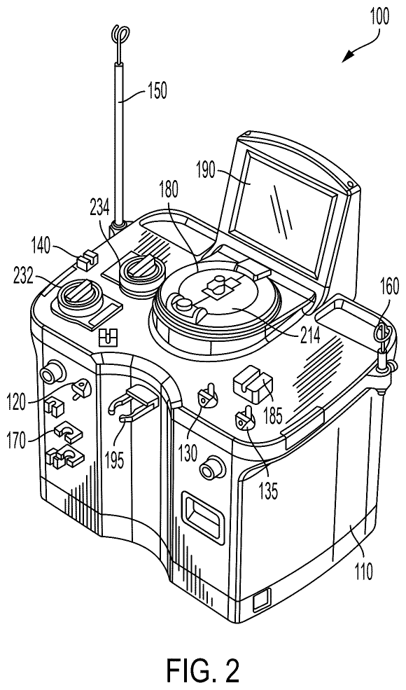

The following FIGS. 2-6 detail some components making up the network 10 and associated network devices as configured in illustrative embodiments and described above with regard to FIG. 1.

Generally speaking, apheresis devices 100 are medical devices designed to selectively remove specific components from a person's blood while returning the remaining blood components back to the individual. As discussed below in greater detail with regard to FIGS. 2-4, the apheresis device 100 has several primary components. First, it incorporates a system for accessing the individual's blood, often through the insertion of intravenous lines or catheters. The device then utilizes various methods, such as centrifugation or filtration, to separate the blood components based on their physical or chemical properties. The desired components are collected into specialized containers or bags for further processing or use. Throughout the process, apheresis devices 100 incorporate monitoring systems and control mechanisms to ensure accuracy and safety. These systems may include sensors, pumps, and software interfaces that regulate flow rates, volumes, and other parameters.

Safety features are a crucial aspect of apheresis devices 100. They can include alarms for pressure or flow irregularities, air detection systems, and safety interlocks to protect the donor, patient, and the operator. A pheresis procedures have a wide range of therapeutic applications, including manufacturing into therapies, collecting blood components for transfusion, removing excess or abnormal substances from the blood, and treating specific medical conditions. Plateletpheresis, plasmapheresis, and leukapheresis are examples of therapeutic apheresis procedures.

In medical settings, such as blood or plasma centers, hospitals, and specialized clinics, as well in mobile settings, apheresis devices 100 are employed by trained professionals to perform these procedures. The devices provide the necessary technology and control to efficiently and safely separate blood components. Their utilization is critical for addressing various therapeutic needs and ensuring the well-being of patients undergoing apheresis procedures.

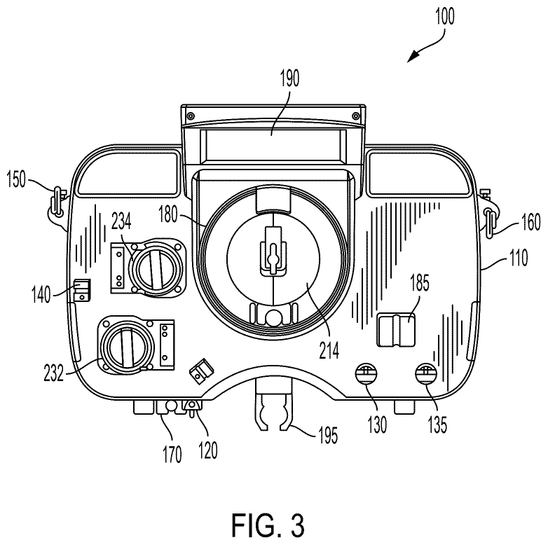

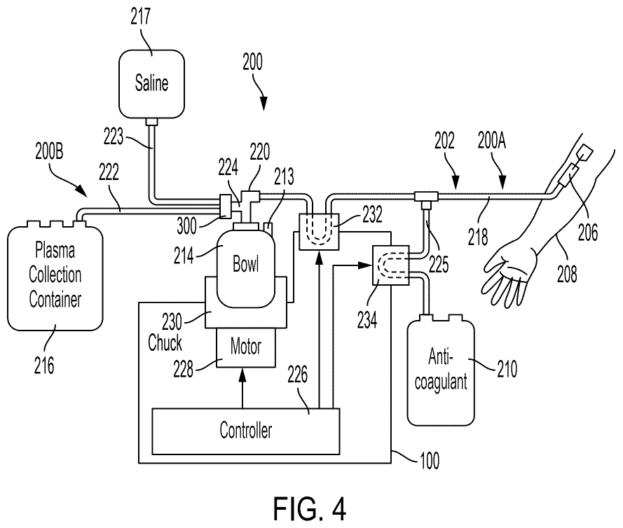

FIG. 2 schematically shows a perspective view of a blood processing system that may be used with illustrative embodiments. FIG. 3 schematically shows a plan view of the blood processing system of FIG. 2. As shown, the blood processing/apheresis device/system 100 includes a cabinet 110 that houses the main components of the system 100 (e.g., the non-disposable components). Within the cabinet 110, the system 100 may include a first/blood pump 232 that draws whole blood from a subject, and a second/anticoagulant pump 234 that pumps anticoagulant through the system 100 and into the drawn whole blood. Additionally, the system 100 may include a number of valves that may be opened and/or closed to control the fluid flow through the system 100. For example, the system 100 may include a donor valve 120 that may open and close to selectively prevent and allow fluid flow through a donor line 218 (e.g., an inlet line shown in FIG. 4), and a plasma valve 130 that selectively prevents and allows fluid flow through an outlet/plasma line 222 (FIG. 4). Some embodiments may also include a saline valve 135 that selectively prevents and allows saline to flow through a saline line 223.

To facilitate the connection and installation of a disposable set and to support the corresponding fluid containers, the system 100 may include an anticoagulant pole 150 on which the anticoagulant solution container 210 (FIG. 4) may be hung, and a saline pole 160 on which a saline solution container 217 (FIG. 4) may be hung (e.g., if the procedure being performed requires the use of saline). Additionally, in some applications, it may be necessary and/or desirable to filter the whole blood drawn from the subject for processing. To that end, the system 100 may include blood filter holder 170 in which the blood filter (located on the disposable set) may be placed.

As discussed in greater detail below, apheresis systems 100 in accordance with illustrative embodiments withdraw whole blood from a subject through a venous access device 206 (FIG. 4) using the blood pump 232. As the system 100 withdraws the whole blood from the subject, the whole blood enters a blood component separation device 214, such as a Latham type centrifuge (other type of separation chambers and devices may be used, such as, without limitation, an integral blow-molded centrifuge bowl, as described in U.S. Pat. Nos. 4,983,158 and 4,943,273). The blood component separation device 214 separates the whole blood into its constituent components (e.g., red blood cells, white blood cell, plasma, and platelets). Accordingly, to facilitate operation of the separation device 214, the system 100 may also include a well 180 in which the separation device 214 may be placed and in which the separation device 214 rotates (e.g., to generate the centrifugal forces required to separate the whole blood).

To allow the user/technician to monitor the system operation and control/set the various parameters of the procedure, the system 100 may include a user interface 190 (e.g., a touch screen device) that displays the operation parameters, any alarm messages, and buttons which the user/technician may depress to control the various parameters. Additional components of the blood processing system 100 are discussed in greater detail below (e.g., in relation to the system operation).

FIG. 4 schematically shows, as a block diagram, the blood processing system 100 and a disposable collection set 200 (with an inlet disposable set 200A and an outlet disposable set 200B) that may be loaded onto/into the blood processing system 100, in accordance with the illustrative embodiments. The collection set 200 includes a venous access device 206 (e.g., a phlebotomy needle) for withdrawing blood from a donor's arm 208, a container of anti-coagulant 210, a centrifugation bowl 214 (e.g., a blood component separation device), a saline container 217, and a final plasma collection bag 216. The blood/inlet line 218 couples the venous access device 206 to an inlet port 220 of the bowl 214, the plasma/outlet line 222 couples an outlet port 224 of the bowl 214 to the plasma collection bag 216, and a saline line 223 connects the outlet port 224 of the bowl 214 to the saline container 217. An anticoagulant line 225 connects the anti-coagulant container 210 to the inlet line 218. In addition to the components mentioned above and as shown in FIG. 4, the blood processing system 100 includes a controller 226, a motor 228, and a centrifuge chuck 230. The controller 226 is operably coupled to the two pumps 232 and 234, and to the motor 228, which, in turn, drives the chuck 230. The controller 226 may be operably coupled to and in communication with the user interface 190.

In operation, the disposable collection set 200 (e.g., the inlet disposable set 200A and the outlet disposable set 200B) may be loaded onto/into the blood processing system 100 prior to blood processing. In particular, the blood/inlet line 218 is routed through the blood/first pump 232 and the anticoagulant line 225 from the anti-coagulant container 210 is routed through the anticoagulant/second pump 234. The centrifugation bowl 214 may then be securely loaded into the chuck 230. After the bowl 214 is secured in place, the technician may install the outlet disposable set 200B. For example, the technician may connect a bowl connector 300 to the outlet 224 of the bowl 214, install the plasma container 216 into the weight senor 195, run the saline line 223 through valve 135, and run the plasma/outlet line 222 through valve 130 and the line sensor 185. After the disposable set 200 is installed and the anticoagulant and saline containers 210/217 are connected, the system 100 is ready to begin blood processing.

A pheresis devices 100 are utilized with donors or patients in a controlled and monitored environment, ensuring the safety and well-being of the individual undergoing the procedure. The process typically begins with careful donor or patient preparation and assessment to determine their eligibility and suitability for apheresis.

Before the procedure, the donor's or patient's vital signs, medical history, and relevant laboratory tests are reviewed to ensure that they meet the specific criteria for apheresis. The donor or patient is informed about the procedure, its purpose, and any potential risks or side effects. Informed consent is obtained to ensure that the donor or patient understands the nature of the procedure and provides their agreement to proceed.

After the donor or patient is prepared, an appropriate blood access point is established to facilitate the collection and return of blood. This may involve the insertion of one or more intravenous lines or catheters, depending on the specific requirements of the apheresis procedure. The access points are carefully chosen to minimize discomfort and ensure adequate blood flow during the process.

Next, the apheresis device 100 is set up and configured based on the prescribed parameters for the procedure. This includes programming the device with the desired settings, such as flow rates, separation protocols, and collection volumes, which are tailored to the individual patient's needs.

During the procedure, the apheresis device 100 carefully extracts blood from the donor or patient through the established blood access point. The blood flows through the device, where, as noted with regard to FIGS. 2-4, it undergoes the separation process, either by centrifugation or filtration. The targeted blood component, such as platelets, plasma, or white blood cells, is selectively collected while the remaining blood components are returned to the patient.

Throughout the procedure, the patient's vital signs, including blood pressure, heart rate, and oxygen saturation, are closely monitored to ensure their safety and well-being. The apheresis device's monitoring systems continuously assess critical parameters, such as flow rates, pressures, and component levels, allowing operators to make real-time adjustments if necessary.

Once the desired amount of the targeted component is collected or the prescribed procedure time is reached, the apheresis device 100 completes the process. The donor's or patient's blood access points are carefully removed, and appropriate post-procedure care and monitoring are provided to ensure their comfort and recovery.

The collected blood components often undergo further processing, testing, and preparation as required for their intended therapeutic use. This can involve additional steps such as component labeling, manufacturing, storage, and compatibility testing to ensure their safety and effectiveness when administered to patients.

As noted, the apheresis devices 100 communicate and are controlled in the network 10 via the noted registration server 14. Unlike a typical network server, the registration server 14 has a special role in the apheresis process. Specifically, the registration server 14 in various embodiments acts as a centralized system that facilitates the registration, monitoring, and/or connectivity of apheresis devices 100 from the same locations and/or remote locations. As such, the registration server 14 streamlines the registration and connectivity process for apheresis devices 100 located at various static and/or mobile sites. It further provides a standardized mechanism for registering and identifying individual devices, allowing users (e.g., providers) to have a comprehensive view of the devices within their network 10.

Operation of the apheresis device 100 may be performed in either disconnected mode or connected mode. In the disconnected mode, the apheresis device 100 does not receive or send data to any network enabled computer software system or device. Instead, data collected by the apheresis device 100 remains on the device itself. In connected mode, however, the apheresis device 100 can receive and send data to network enabled computer software systems or devices. The flow of data is bi-directional, meaning it can both send and receive data from external systems.

As noted above, apheresis devices 100 from a single or related unit (e.g., a plasma center) can be physically located in different facilities or clinical settings. The registration server 14 therefore acts as a central hub that enables remote management and monitoring of these devices. In addition, the registration server 14 may track assets and manage device activation in case devices 100 are lost or stolen. Registration may involve receiving device-specific information, such as model numbers, activation codes, license information, unique identifiers, and location details. The registration process ensures that each device is properly activated, licensed, authorized, and integrated into the software system, facilitating seamless communication and control.

Importantly, the registration server 14 enables remote connectivity and communication with registered apheresis devices 100. It provides a secure and reliable means for remote operators or administrators to access and manage the devices. Through this connectivity, operators can monitor device status, review operational parameters, and perform remote troubleshooting or maintenance tasks as needed. The registration server 14 thus can act as the bridge that facilitates the exchange of data and commands between the remote operators and the apheresis devices 100.

The registration server 14 can be implemented in different ways, depending on the specific setup and requirements of the apheresis devices 100 and the organization. For example, the registration server 14 can be located on the same site as the apheresis devices 100, remotely hosted in an off premises (e.g., in a secure data center), or a combination of both. On-site implementations can facilitate direct and immediate access to these and other devices from the local network 10. Operators or administrators can register and manage the devices within the same physical location, facilitating efficient communication and control. In certain scenarios, a combination of both local and remote configurations may be implemented. This hybrid approach may involve a local registration server 14 at each site where apheresis devices 100 are present, while also connecting to a central remote registration server 14 for higher-level management and coordination. In some embodiments, this setup allows for a balance between local control and centralized oversight. Other embodiments facilitate centralized oversight of the various devices 100 (e.g., a single entity having oversight of many devices 100 across may locations).

The choice of deployment configuration depends on factors, such as the number and distribution of apheresis devices 100, licensing and activation requirements, the organization's IT infrastructure, connectivity requirements, and security considerations. One goal is to ensure seamless connectivity, effective device management, and reliable access to the apheresis devices 100, regardless of whether the registration server 14 is on-site, remote, or a combination of both.

The apheresis device 100 preferably is configured to operate in the connected mode and to automatically connect with the registration server 14. When the apheresis device 100 connects to the Internet (whether through the noted cellular connection or some other connection), it will automatically register itself with the registration server 14 by sending data about itself, including specific identification data such as serial identifier, location, timestamps, component versions, or any relevant device data such as procedure data, user, or location specific information. After receipt, the registration server 14 preferably processes, stores, and provides a response to the apheresis device 100. This response may include a simple confirmation of device registration, activation codes, licenses, delivery of update files, programming instructions, or new messages received from an external network-enabled system, such as the noted BECS 12 or another networked device. A BECS 12 is shown here as one exemplary implementation.

As known by those in the art, the BECS 12 typically plays an important role in the optimization of blood banking and plasma collection operations. Designed specifically for blood establishments, these specialized systems offer comprehensive functionalities to manage the collection, testing, processing, storage, and distribution of blood and blood products. When appropriately configured, the BECS 12 can often ensure the safety, traceability, and efficiency of the blood and blood products supply chain.

An important aspect of the BECS 12 in some embodiments involves donor management. This device favorably facilitates the registration and screening of donors, capturing crucial demographic information and medical history. It tracks the lifecycle of a donor's engagement, including donation records, deferrals, and notifications for repeat donations. With robust donor management capabilities, blood establishments can maintain accurate and up-to-date donor data, ensuring the eligibility and safety of the donated blood products.

Another important structure within the BECS 12 involves component management. This functionality allows blood establishments to track the processing and manufacturing of various blood components, such as red blood cells, platelets, plasma, and cryoprecipitate. By closely monitoring the inventory and location of these components, the BECS 12 enables efficient allocation and usage, minimizing waste and ensuring optimal stock rotation. Additionally, component management ensures proper labeling and tracking, enabling swift retrieval and traceability when needed.

Quality control and testing are integral parts of any blood banking operation, and the BECS 12 streamlines these processes. The system typically integrates laboratory testing results, including compatibility testing, infectious disease screening, and component quality assessment. Test results are securely stored within the BECS 12, facilitating quick access and retrieval when needed. Moreover, the system manages the inventory of test kits and reagents, ensuring their availability and tracking expiration dates. By automating testing and quality control procedures, the BECS 12 enhances accuracy, efficiency, and regulatory compliance.

Reporting and regulatory compliance also form a critical aspect of blood banking operations. To that end, the BECS 12 generates a wide range of reports, including blood inventory, donor statistics, adverse events, and performance indicators. These reports not only provide valuable insights for internal management, while also aiding in complying with national and international standards and guidelines. The BECS 12 thus serves as a reliable tool for blood and plasma collection establishments to demonstrate adherence to regulatory requirements and continuously monitor and improve their operations. Some of these functions can cooperate with the apheresis device 100 and registration server 14 to appropriately and effectively complete blood donation and processing.

In the embodiment shown, the registration server 14 acts as a middleware type software element (“middleware”) in the network 10 between the BECS 12 and the apheresis device 100. To that end, the registration server 14 preferably has a middleware engine acting as a bridge, facilitating communication and data exchange between the apheresis devices 100 and the BECS 12. As known by those in the art, middleware engines (also known as “interface engines”) are configured to handle data integration and interoperability between different systems that may use different communication protocols or have incompatible interfaces. They act as a mediator, translating the data formats, protocols, and commands used by the BECS 12 and the apheresis device 100, enabling seamless communication.

Some embodiments may use one or more of the following steps when employing middleware or interface engines:
Integration Configuration: The middleware or interface engine is configured to understand the data formats and communication protocols used by both the BECS 12 and the apheresis device 100. This configuration allows the middleware to act as a translator between the two systems.
Data Translation: The middleware translates the data from the BECS 12 into a format that the apheresis device 100 can understand, and vice versa. This involves mapping the data elements, converting data structures, and ensuring compatibility between the systems.
Protocol Conversion: If the BECS 12 and the apheresis device 100 use different communication protocols, the middleware converts the data transmission to align with the protocols supported by both systems. This ensures that the data can be successfully transmitted and received by each system.
Command Routing: The middleware receives commands from the BECS 12 and routes them to the apheresis device 100. It ensures that the commands are properly formatted and translated to the appropriate commands understood by the apheresis device 100.

Similarly, the middleware receives data and status updates from the apheresis device 100 and forwards them to the BECS 12.

By utilizing a middleware or interface engine, blood establishments can achieve interoperability between their BECS 12 and apheresis devices 100 that may have different communication capabilities or protocols. This approach enables seamless data transfer, command execution, and status monitoring between the two systems.

Middleware or interface engines often adhere to industry standards for healthcare data exchange, such as Health Level Seven (HL7) or Integrating the Healthcare Enterprise (IHE). These standards ensure compatibility and interoperability across different systems within the healthcare domain, facilitating smooth integration and communication between the BECS 12 and the apheresis device 100.

Alternative embodiments may use other approaches for interconnecting the elements of the network 10. For example, in some cases, the BECS 12 may have built-in capabilities to directly integrate with compatible apheresis devices 100. This integration may be achieved through standardized communication protocols or interfaces. The BECS 12 sends commands and receives data directly from the apheresis device 100, allowing for seamless data exchange and control. Other embodiments may have device-specific interfaces. For example, apheresis devices 100 often have their own software interfaces or application programming interfaces (A Pls) that enable communication with external systems like the BECS 12. These interfaces provide a standardized way for the BECS 12 to interact with the apheresis device 100, allowing for data transfer, command execution, and status updates. In either case, some embodiments provide for the desired plug-and-play capability for the apheresis devices 100.

Accordingly, discussion of the specific interconnections, as well as some of the devices above (e.g., the BECS 12) are illustrative and not intended to limit various embodiments of the invention.

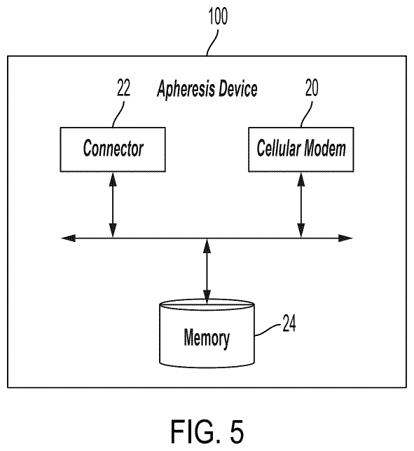

FIG. 5 schematically shows selected parts of an apheresis device 100 configured in accordance with illustrative embodiments. Each of these components is operatively connected by any conventional interconnect mechanism. As shown in FIG. 5, a bus communicates between components described below. Those skilled in the art should understand that this generalized representation can be modified to include other conventional direct or indirect connections. Accordingly, discussion of a bus is not intended to limit various embodiments.

Indeed, FIG. 5 schematically shows example selected components/parts of the apheresis device 100. Those skilled in the art should understand that each of these components can be implemented in a variety of conventional manners, such as by using hardware, software, or a combination of hardware and software, across one or more other functional components. For example, the connector (to be discussed) may be implemented using a plurality of microprocessors executing firmware. As another example, the connector may be implemented using one or more application specific integrated circuits (i.e., “ASICs”) and related software, or a combination of ASICS, discrete electronic components (e.g., transistors), and microprocessors. Accordingly, the representation of the connector and other components in a single box of FIG. 5 is for simplicity purposes only. In fact, in some embodiments, the connector or other parts shown in FIG. 5 can be distributed across a plurality of different machines—not necessarily within the same housing or chassis.

It should be reiterated that the representation of FIG. 5 is a significantly simplified representation of an apheresis device 100. Those skilled in the art should understand that such a device has many other physical and functional components, such as those discussed above with regard to FIGS. 2-4, along with central processing units, other communication processing modules, and short-term memory. Accordingly, this discussion is not intended to suggest that FIG. 5 represents all of the elements of the apheresis device 100.

As shown, the apheresis device 100 has long term memory 24 for storing information (e.g., solid state memory), such as instructions and data from the BECS 12 and donor or patient data, a cellular modem 20 for communicating with remote devices, such as the registration server 14, and a connector 22 configured to detect (e.g., when powered on searching for a signal) the cellular connection with the apheresis device 100.

As noted, the connector 22 also is configured to automatically communicate, via the cellular connection, with the registration server 14 after detecting the cellular connection. This automatically initiates communication and a corresponding an apheresis registration process with the registration server 14. Specifically, as an automatic process, it may be begun and completed with little or no direct human or manual control. Instead, the connector 22 is configured with the functionality to communicate with the registration server 14, exchange handshake and registration information, and coordinate a data/network connection.

In various embodiments, the connector 22 may utilize 3GPP standard registration protocol to establish a radio resource control (RRC) connected status with the RAN to communicate with the server 14.

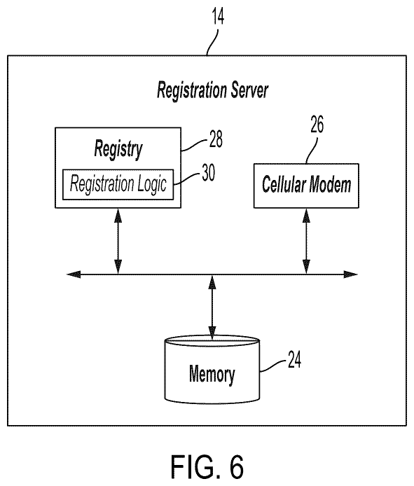

FIG. 6 schematically shows selected parts of the registration server 14 configured in accordance with illustrative embodiments. This registration server 14 may interact with the apheresis device 100 via the cellular or other network connection to automatically register the apheresis device 100 upon connection of the apheresis device 100. Among other things, as known by those in the art, to register, the system ensures that the device 100 is valid, licensed, and intended for use to whom it was sold or placed. Like the technology of FIG. 6, those skilled in the art should understand that each of these components can be implemented in a variety of conventional manners, such as by using hardware, software, or a combination of hardware and software, across one or more other functional components. The above examples used for FIG. 5 may apply to the registration server 14, such as the different implementations in hardware/software/firmware etc., in a plurality of parts across different machines or the same machine, its significant simplification for discussion purposes, etc. Moreover, the interconnect mechanism (i.e., the bus shown in FIG. 6) also has the same qualifications as those discussed above with regard to FIG. 5.

As shown, the registration server 14 also has long-term memory 24 for storing information, such as activation codes, licenses, instructions and data from the apheresis device 100 and/or the BECS 12, as well as donor or patient data and other configuration information. The registration server 14 further has a cellular modem 26 for communicating with remote devices, such as the BECS 12 and the apheresis device 100. Importantly, the registration server 14 also has an apheresis device registry 28 with registration logic 30 configured to automatically register one or more apheresis devices 100 with authorization to join the network. In some embodiments, if the apheresis device 100 is not authorized to join the network, then it may be “bricked”. That is, the apheresis device 100 will be unable to perform its procedures without first joining the network.

Similarly to the apheresis device 100 described above, in various embodiments, the registration server 14 may utilize 3GPP standard registration protocol to establish a RRC connected status with the RAN to communicate with the apheresis device 100.

FIG. 7 shows a process of registering an apheresis device 100 in accordance with illustrative embodiments. It should be noted that this process is substantially simplified from a longer process that normally would be used to register an apheresis device 100. Accordingly, the registration process has many steps that those skilled in the art likely would use. In addition, some of the steps may be performed in a different order than that shown, or at the same time. Those skilled in the art therefore can modify the process as appropriate. Moreover, as noted above and below, the specific processes and structures in each step are exemplary. As such, those skilled in the art can use other processes and/or structures to accomplish the goal of one or more steps, depending upon the application and other constraints. Accordingly, discussion of specific processes and structures is not intended to limit all embodiments.

The process of FIG. 7 begins at step 700 in which the apheresis device 100 initiates communication with the network 10—the beginning of the connected mode. In accordance with illustrative embodiments, this happens when the apheresis device 100 uses conventional or proprietary techniques to request access to the network 10 of FIG. 1. In the embodiment of FIG. 1, the cellular modem 20 of the apheresis device 100 communicates, via the network 10, with the cellular modem 26 or, in other embodiments, another interface of the registration server 14 (e.g., an Ethernet or Wi-Fi connection of the registration server 14). This may be accomplished by use of intermediate cell towers (shown by example), satellites, and the Internet/cloud between the two devices 100 and 14 (e.g., peer-to-peer, shared, Internet connection, or hot-spotting).

Connecting to a cellular network 10 typically involves a series of sequential steps designed to ensure proper registration (not the same registration as with the registration server 14) and authentication. When the apheresis device 100 is powered on or wakes up from a sleep state, its cellular modem 20 becomes active and it begins scanning for available cellular networks in the vicinity. This step, known as network selection, evaluates signal strength, signal quality, and other parameters of nearby base stations to determine the most suitable one. As mentioned above, the apheresis device may establish an RRC connection with the RAN via the base station.

For example, after identifying a suitable base station, the device initiates a registration request to the cellular network 10. In some embodiments, during RRC registration it may send its International Mobile Equipment Identity (IMEI) and International Mobile Subscriber Identity (IM SI) or Subscription Permanent Identifier (SUPI) to the network 10. The network 10 validates the device's identity and authorization by verifying the IMEI and IMSI or SUPI, ensuring it is a legitimate and authorized device.

Upon successful authentication, the network 10 may assign a Temporary Mobile Subscriber Identity (TMSI) to the device for enhanced security and privacy. This temporary identifier is used during communications with the network 10 to prevent the device's actual IMSI from being transmitted over the air frequently. Additionally, the device provides its location information to the network 10 in a location update process, allowing the network 10 to know the general location of the device within its coverage area. Some embodiments also validate that the device 100 is operating in a licensed/authorized geographic area. For example, a device 100 sold in the US may have a restriction that it cannot be shipped/used in Asia. This is important for theft/loss prevention, tracking, and also for international law certification requirements.

After the apheresis device 100 is authenticated and registered, the network 10 activates the requested services, such as data services and messaging. With proper registration and service activation, the apheresis device 100 may enter an idle state, ready to send or receive data or initiate voice calls whenever necessary. Preferably, however, the blood process network registration process begins automatically and immediately after activation. Throughout the connection process, the cellular modem 20 may continuously monitor its connection status and signal strength. If the connection is lost or the signal becomes weak, the cellular modem 20 may attempt to re-register with another base station to maintain a stable and reliable connection to the cellular network 10.

At this point, the connector 22 detects this cellular connection (step 702) and in response, automatically begins registering with the registration server 14. As noted, this registration process may involve forwarding handshake registration information related to a specific connection/communication protocol from the apheresis device 100 toward the registration server 14. This also may include registration/device information about the apheresis device 100 itself (step 704). As noted above, this registration information may include information about the apheresis device 100, including specific identification data such as serial identifier, location, timestamps, component versions, or any relevant device data such as procedure data, user, or location specific information.

In response to receipt of the registration information, the registration server 14 may validate the specific apheresis device 100 and return registration information or other information to the requesting apheresis device 100 (step 706). Such return registration information may be specific to the application programming interface (API), or communication protocol used by the network 10. In some embodiments, the registration server 14 checks the specific identification data to ensure both security and that the apheresis has authorization to register with the network 10. For example, the registration server 14 may confirm that the location of the apheresis device 100 is the same as that in its database of approved locations. As another example, the registration server 14 may confirm the model number or serial number of the apheresis device 100 to confirm it is supported or matches the device ownership information.

There may be instances where validation fails. In that case, in some embodiments, the device 100 may react or operate in some preconfigured manner. For example, the device may be permanently or temporarily disabled—i.e., made to be non-functional. In other embodiments, it may send a signal to a central authority with certain information (e.g., license information, location information, information indicating an error condition, etc.).

As noted above, while the above process essentially is a “plug-and-play” process, some embodiments may require minimal user interaction and still be considered essentially automatic. For example, that minimal interaction may include approving the registration process moving forward, or entering an authentication identification from the operator connecting the apheresis device 100.

Either way, in illustrative embodiments, the apheresis device 100 can be connected to the network 10 and start working automatically at startup or restart (or at other selected times) without the need for manual configuration or additional software installation. As such, it is configured to be easily recognized and integrated into the network infrastructure seamlessly. In this case, the connector 22 automatically detects the presence of the network 10 via the cellular connection and enables/initiates the registration functionality. This may involve use of built-in drivers, or even installation of other necessary drivers or software to enable its functionality. Preferably, this allows users to simply plug in the apheresis device 100, start the cellular modem 20 (or it may automatically start when the apheresis device 100 powers up), and it will be ready to use quickly without requiring material technical expertise or intervention.

Accordingly, after completing the process of FIG. 7, the apheresis device 100 may be used with donors or patients to collect blood and produce the requisite blood components. Importantly, as noted above in one example, modification, swap outs/replacements (in small or large numbers), or updates of an entire plasma center with significant numbers of apheresis devices 100 may be more easily updated in a much more expedited and less costly manner. Use of the cellular modem 20 further enables a portable architecture for enhanced use cases, such as outside of a traditional brick-and-mortar plasma center (e.g., in a rural location or a stadium), or without being constrained to the layout of the plasma center and its network wiring, network configuration, and/or signal strength.

The operations described above in FIG. 7 are exemplary and may be performed in different orders, or some operations not performed, in various embodiments. Various references to protocols for cellular communication are utilized above that may refer to operations and technical aspects relating to a particular radio access technology (e.g., long term evolution (LTE), 5G, 6G, 802.xx, etc.). However, other radio access technologies may be utilized, along with their associated protocols, signaling and establishment of radio connections.

FIG. 8 is a diagram of an example embodiment of signals and operations among an apheresis system, a registration and connectivity computer software server and a BECS in accordance with illustrative embodiments. In various embodiments, the components depicted in FIG. 8 may correspond to similar components described above in FIGS. 1, 5 and 6 for example. In various embodiments, the. It will be understood that a described signal may have associated operations and a described operation may have associated signals.

In various embodiments, components performing the operations of FIG. 8 may be a same device or reside in a same device. In various embodiments, components performing the operations of FIG. 8 may reside in separate devices from one another.

At operation 801, the apheresis system receives registration information. In various embodiments, the registration information may be received from one or more apheresis devices.

Accordingly, at operation 802, the apheresis system transmits a registration information message to the registration and connectivity computer software server and the registration and connectivity computer software server receives the registration information message. In various embodiments, the registration information message includes information about the apheresis device 100, including specific identification data such as serial identifier, location, timestamps, component versions, or any relevant device data such as procedure data, user, or location specific information.

Once the registration and connectivity computer software server receives the registration information, the registration and connectivity computer software server may validate the apheresis device at operation 803. In various embodiments, the validation may include ensuring that the apheresis device is valid, licensed, and intended for use to whom it was sold or placed. In various embodiments, the validation may include determining that a geographic area of use is valid for the apheresis device. Additional validation embodiments are described above.

At operation 804, the registration and connectivity computer software server transmits a registration response to the apheresis system and the apheresis system receives the registration response. As discussed above, the registration response may include a confirmation of device registration, activation codes, licenses, delivery of update files, programming instructions, or new messages received from an external network-enabled system, such as the noted BECS or another networked device.

Once the apheresis system receives the registration response message, the apheresis system may establish communication (communication may occur) with the BECS at operation 805. In various embodiments, the communication may occur directly with the BECS or may be an indirect communication through another entity.

The operations of FIG. 8 are merely illustrative, and variations are contemplated to be within the scope of the present disclosure. In embodiments, the operations may include other operations not illustrated in FIG. 8. In embodiments, the operations may not include every operation illustrated in FIG. 8. In embodiments, the operations may be implemented in a different order than that illustrated in FIG. 8. Such and other embodiments are contemplated to be within the scope of the present disclosure. Persons of skill in the art will appreciate that, although various example components are described as perform various functions, other components may perform those functions described in FIG. 8.

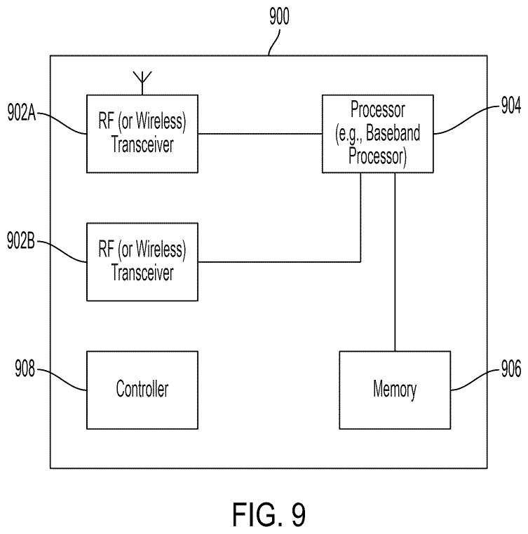

FIG. 9 is an example block diagram of a device 900 in accordance with illustrative embodiments. In various embodiments, the device 900 may be any one of the devices depicted in FIGS. 1, 5 and 6 and may be a wired or wireless device. For purposes of example, device 900 is depicted as a wireless device in FIG. 9.

In various embodiments, the device 900 may include, for example, one or more (e.g., two as shown in FIG. 9) RF (radio frequency) or wireless transceivers 902A, 902B, where each wireless transceiver includes a transmitter to transmit signals and a receiver to receive signals. The device 900 also includes a processor or control unit/entity (controller) 904 to execute instructions or software and control transmission and receptions of signals, and a memory 906 to store data and/or instructions.

Processor 904 may also make decisions or determinations, generate frames, packets or messages for transmission, decode received frames or messages for further processing, and other tasks or functions described herein. Processor 904, which may be a baseband processor, for example, may generate messages, packets, frames or other signals for transmission via wireless transceiver 902 (902A or 902B). Processor 904 may control transmission of signals or messages over a wireless network, and may control the reception of signals or messages, etc., via a wireless network (e.g., after being down-converted by wireless transceiver 902, for example). Processor 904 may be programmable and capable of executing software or other instructions stored in memory or on other computer media to perform the various tasks and functions described above, such as one or more of the tasks or methods described above. Processor 904 may be (or may include), for example, hardware, programmable logic, a programmable processor that executes software or firmware, and/or any combination of these. Using other terminology, processor 904 and transceiver 902 together may be considered as a wireless transmitter/receiver system, for example.

In addition, referring to FIG. 9, a controller (or processor) 908 may execute software and instructions, and may provide overall control for the station 900, and may provide control for other systems not shown in FIG. 9, such as controlling input/output devices (e.g., display, keypad), and/or may execute software for one or more applications that may be provided on wireless device 900, such as, for example, an email program, audio/video applications, a word processor, a Voice over IP application, or other application or software.

In addition, a storage medium may be provided that includes stored instructions, which when executed by a controller or processor may result in the processor 904, or other controller or processor, performing one or more of the functions or tasks described above.

According to another example embodiment, RF or wireless transceiver(s) 902A/902B may receive signals or data and/or transmit or send signals or data. Processor 904 (and possibly transceivers 902A/902B) may control the RF or wireless transceiver 902A or 902B to receive, send, broadcast or transmit signals or data.

Example embodiments are provided or described for each of the example methods, including: An apparatus (e.g., 900, FIG. 9) including means (e.g., processor 904, RF transceivers 902A and/or 902B, and/or memory 906, in FIG. 9) for carrying out any of the methods; a non-transitory computer-readable storage medium (e.g., memory 906, FIG. 9) comprising instructions stored thereon that, when executed by at least one processor (processor 904, FIG. 9), are configured to cause a computing system (e.g., 900, FIG. 9) to perform any of the example methods; and an apparatus (e.g., 900, FIG. 9) including at least one processor (e.g., processor 904, FIG. 9), and at least one memory (e.g., memory 906, FIG. 9) including computer program code, the at least one memory (906) and the computer program code configured to, with the at least one processor (904), cause the apparatus (e.g., 900) at least to perform any of the example methods.

Various embodiments of the invention may be implemented at least in part in any conventional computer programming language. For example, some embodiments may be implemented in a procedural programming language (e.g., “C”), or in an object-oriented programming language (e.g., “C++”). Other embodiments of the invention may be implemented as a pre-configured, stand-alone hardware element and/or as preprogrammed hardware elements (e.g., application specific integrated circuits, FPGAS, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, solid state drive, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments of the invention may be implemented as a combination of both software (e.g., a computer program product) and hardware. Still other embodiments of the invention are implemented as entirely hardware, or entirely software.

The embodiments of the invention described above are intended to be merely exemplary; numerous variations and modifications will be apparent to those skilled in the art. Such variations and modifications are intended to be within the scope of the various embodiments. Note that the below sentences referred to as “claims” are innovative concepts and may vary from any claims in an application claiming priority hereto.

What is claimed is:

1. A portable apheresis system for separating blood into blood components, the apheresis system comprising:
a cellular modem configured to transmit and receive information to and from the apheresis device via a cellular connection; and
a connector operatively coupled with the cellular modem, the connector configured to control the cellular modem to transmit and receive apheresis device registration information, the connector also configured to:
establish, using the cellular modem, a cellular communication connection between an apheresis device and a cellular network after detecting the cellular network, transmit registration information relating to the apheresis device to a registration and connectivity computer software server via the wireless communication connection,
receive an apheresis registration response from the registration and connectivity computer software server, the apheresis registration response including information relating to the apheresis device registration process, the registration process causing direct or indirect apheresis device communication with a blood establishment computer system (BECS) remotely positioned relative to the apheresis device.

2. The system of claim 1, wherein the registration information comprises one or more of a serial identifier, location information, timestamps, component versions, procedure data, user information, a device activation code, licensing, authorized use information, or location specific information.

3. The system of claim 1, wherein the apheresis device is validated based upon one or more of the following: the apheresis device is valid, licensed, and intended for use to whom it was sold or placed, or determining that a geographic area of use is valid for the apheresis device.

4. The system of claim 3, wherein the connector is configured to receive registration confirmation information from the registration and connectivity computer software server via the cellular connection, the apheresis device being prevented from operating when validation fails or when the apheresis device is not registered.

5. The system of claim 1, wherein the registration information includes one or more of the following: information about the apheresis device, identification data or device data.

6. The system of claim 5, wherein the identification data includes one or more of the following: a serial identifier, location, timestamps, or component versions.

7. The system of claim 5, wherein the device data includes one or more of the following: procedure data, user data or location specific information.

8. The system of claim 1, wherein the registration process is plug-and-play.

9. The system of claim 1, further comprising a mobile platform containing the apheresis device, the mobile platform being towable or self-propelled.

10. A method for separating blood into blood components, the method comprising:
transmitting and receiving, via a cellular modem, information to and from the apheresis device via a cellular connection;
controlling the cellular modem to transmit and receive apheresis device registration information;
establishing, by the connector, using the cellular modem, a cellular communication connection between the apheresis device and a cellular network after detecting the cellular network;
transmitting registration information relating to the apheresis device to a registration and connectivity computer software server via the wireless communication connection; and
receiving an apheresis registration response from the registration and connectivity computer software server, the apheresis registration response including information relating to the apheresis device registration process, the registration process causing direct or indirect apheresis device communication with a blood establishment computer system (BECS) remotely positioned relative to the apheresis device.

11. The method of claim 10, wherein the registration information comprises one or more of a serial identifier, location information, timestamps, component versions, procedure data, user information, a device activation code, licensing, authorized use information, or location specific information.

12. The method of claim 10, further comprising validating, by the registration and connectivity computer software server based upon one or more of the following: the apheresis device is valid, licensed, and intended for use to whom it was sold or placed, or determining that a geographic area of use is valid for the apheresis device.

13. The method of claim 12, wherein the registration response is received from the registration and connectivity computer software server via the cellular connection, the apheresis device being prevented from operating when validation fails or when the apheresis device is not registered.

14. The method of claim 10, wherein the registration information includes one or more of the following: information about the apheresis device, identification data or device data.

15. The method of claim 14, wherein the identification data includes one or more of the following: a serial identifier, location, timestamps, or component versions.

16. The method of claim 14, wherein the device data includes one or more of the following: procedure data, user data or location specific information.

17. A computer program product for use on a computer system, the computer program product comprising a tangible, non-transient computer usable medium having computer readable program code thereon, the computer readable program code comprising:
program code for transmitting and receiving, via a cellular modem, information to and from the apheresis device via a cellular connection;
program code for controlling the cellular modem to transmit and receive apheresis device registration information;
program code for establishing, by the connector, using the cellular modem, a cellular communication connection between the apheresis device and a cellular network after detecting the cellular network;
program code for transmitting registration information relating to the apheresis device to a registration and connectivity computer software server via the wireless communication connection; and
program code for receiving an apheresis registration response from the registration and connectivity computer software server, the apheresis registration response including information relating to the apheresis device registration process, the registration process causing direct or indirect apheresis device communication with a blood establishment computer system (BECS) remotely positioned relative to the apheresis device.

18. The computer program product of claim 17, further comprising program code for validating, by the registration and connectivity computer software server based upon one or more of the following: the apheresis device is valid, licensed, and intended for use to whom it was sold or placed, or determining that a geographic area of use is valid for the apheresis device.

19. The computer program product of claim 18, wherein the registration response is received from the registration and connectivity computer software server via the cellular connection, the apheresis device being prevented from operating when validation fails or when the apheresis device is not registered.

20. The computer program product of claim 17, wherein the registration information includes one or more of the following: information about the apheresis device, identification data or device data.
